(19) United States
(12) Patent Application Publication
(10) Pub. No.: US 2016/0067393 A1
(43) Pub. Date: Mar. 10, 2016

(54) SYSTEM AND METHOD OF USE FOR A CONCEALABLE AND REDUCED NOISE BREAST PUMP

(71) Applicant: Tylane Barnes, San Antonio, TX (US)

(72) Inventor: Tylane Barnes, San Antonio, TX (US)

(21) Appl. No.: 14/846,669

(22) Filed: Sep. 4, 2015

Related U.S. Application Data
(60) Provisional application No. 62/046,131, filed on Sep. 4, 2014.

(51) Int. Cl. A61M 1/06; A61J 13/00

(52) U.S. Cl. CPC A61M 1/06; A61M 1/062; A61J 13/00; A61M 2210/1007; A61M 2205/8206

(57) ABSTRACT

A system and method of use for a concealable breast pump is disclosed herein. The concealable and reduced noise breast pump generally is comprised of a breast shield member, a diaphragm member, a fluid capture member, a power supply member, a motor member, a pump member, and connection tube members. In an embodiment, the connection tube members join the breast shield member, the diaphragm member, and the pump member to each other. In another embodiment the concealable and reduced noise breast pump is further comprised of belt to be worn around the waist of a user of the system which allows certain members such as the diaphragm member, the fluid capture member, the power supply member, the motor member, and/or the pump members to be releasably connected to the belt member. In yet another embodiment, the system is further comprised of noise dampening members, which cover certain members of the system to reduce the noise level heard from the system.

### CROSS-REFERENCE TO RELATED APPLICATIONS

This application claims the benefit under Title 35 United States Code §119(e) of U.S. Provisional Patent Application Ser. No. 62/046,131; Filed: Sep. 4, 2015, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates generally to breast pumping systems and their methods of use. More specifically, the present invention relates to breast pumping systems that are portable and their methods of use.

### BACKGROUND OF THE INVENTION

Without limiting the scope of the disclosed system and method, the background is described in connection with a novel system and method directed to a concealable and reduced noise breast pump.

The field's prior art reflects many approaches and devices in providing a breast pump. However, the conventional approaches employed have several drawbacks. As a first example, commonly used breast pumps are either manual or contain a noisy motor. In addition, many commonly used breast pumps are utilized in a viewable manner. That is, the user of the breast pump is exposed or the breast pump itself is exposed visually to others.

Due to these characteristics of existing breast pumps, the user often has to find a discrete location to extract her milk using these existing devices.

In view of the foregoing, it is apparent that there exists a need in the art for a concealable and reduced noise breast pump, which overcomes, mitigates, or solves the above problems in the art. It is the purpose of this invention to fulfill this and other needs in the art, which will become apparent to the skilled artisan once given the following disclosure.

### BRIEF SUMMARY OF THE INVENTION

The present invention, therefore, provides for a system and method of use for a concealable and reduced noise breast pump.

In one embodiment, the concealable and reduced noise breast pump is comprised of a breast shield member, a diaphragm member, a fluid capture member, a power supply member, a motor member, a pump member, and connection tube members. In an embodiment, the connection tube members join the breast shield member, the diaphragm member, and the pump member to each other. In another embodiment the concealable and reduced noise breast pump is further comprised of a belt to be worn around the waist of a user of the system which allows certain members such as the diaphragm member, the fluid capture member, the power supply member, the motor member, and/or the pump members to be releasably connected to the belt member. In yet another embodiment, the system is further comprised of noise dampening members, which cover certain members of the system to reduce the noise level heard from the system

In summary, the present invention relates generally to breast pumping systems and their methods of use. More specifically, the present invention relates to breast pumping systems that are portable, concealable, and have reduced noise characteristics. In addition, their methods of use are also described and claimed.

### BRIEF DESCRIPTION OF THE SEVERAL VIEWS OF THE DRAWINGS

The accompanying drawings, which are incorporated in and form a part of the specification, illustrate a preferred embodiment of the present invention, and together with the description, serve to explain the principles of the invention. It is to be expressly understood that the drawings are for the purpose of illustration and description only and are not intended as a definition of the limits of the invention. In the drawings:

FIG. 1 is a left side perspective view of the concealable and reduced noise breast pump in accordance with the teachings of the present disclosure;

FIG. 2 is a left side view of the concealable and reduced noise breast pump in accordance with the teachings of the present disclosure;

FIG. 3 is a right side view of the concealable and reduced noise breast pump in accordance with the teachings of the present disclosure;

FIG. 4 is a front view of the concealable and reduced noise breast pump in accordance with the teachings of the present disclosure;

FIG. 5 is a back view of the concealable and reduced noise breast pump in accordance with the teachings of the present disclosure;

FIG. 6 is a top view of the concealable and reduced noise breast pump in accordance with the teachings of the present disclosure;

FIG. 7 is a bottom view of the concealable and reduced noise breast pump in accordance with the teachings of the present disclosure;

FIG. 8 is a left side perspective view of the concealable and reduced noise breast pump with noise dampening insulation in accordance with the teachings of the present disclosure;

FIG. 9 is a left side perspective view of the concealable and reduced noise breast pump with noise dampening insulation and attachment belt in accordance with the teachings of the present disclosure;

FIG. 10 is an environmental view of the concealable and reduced noise breast pump with noise dampening insulation and attachment belt being utilized by a user in accordance with the teachings of the present disclosure.

### DETAILED DESCRIPTION OF THE INVENTION

Disclosed herein is an improved system and method of use for a concealable and reduced noise breast pump. The numerous innovative teachings of the present invention will be described with particular reference to several embodiments (by way of example, and not of limitation).

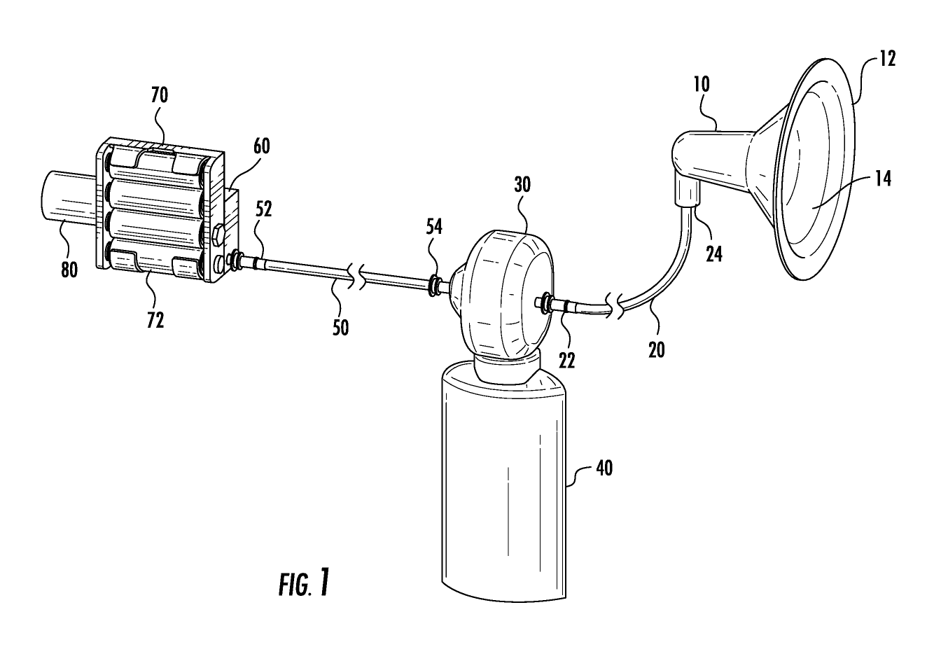

Reference is first made to FIG. 1, a left side perspective view of the concealable and reduced noise breast pump in accordance with the teachings of the present disclosure. Illustrated in this figure are the components that make up the concealable and reduced noise breast pump. In one embodiment, the concealable and reduced noise breast pump is comprised of a breast shield member 10, a diaphragm member 30, a fluid capture member 40, a power supply member 70, a motor member 80, a pump member 60, and connection tube members 20, 50.

In an embodiment, the front connection tube member 20 joins the breast shield member 10 with the diaphragm member 30 and the back connection tube member 50 joins the diaphragm member 30 with the pump member 60. The breast milk being pumped from the user's breast flows through the breast shield member 10, through the front connection tube member 20, through the diaphragm member 30, and into the fluid capture member 40. The connection tube members 20, 50 may have various lengths to assist in the positioning and fitting of the components on the user. For example, connection tube member 20 may have a length allowing the connection tube member 20 to reach from the diaphragm member 30 positioned at the waist of the user to the breast shield member 10 positioned on the user's breast. In an embodiment, the connection tube members 20, 50 are made of flexible tubing such as a rubber or polymer. In another embodiment, the connection tube members 20, 50 are made out of a tubing material that allows elongation and retraction. An example of this material is elastic materials such as a polymer that allows stretching. In yet another embodiment, the connection tube members 20, 50 are comprised of connecting portions 22, 24, 52, 54. The connection portions 22, 24, 52, 54 allow the connection tube members 20, 50 to be attached and detached at their ends 22, 24, 52, 54. For example, the front connection tube member 20 may be attached and detached from the diaphragm member 30 with the diaphragm connection portion 22. In an embodiment, there is no connection portion at the connection point 24 between the front connection tube member 20 and the breast shield member 10. In another embodiment, there is a connection portion at the connection point 24 between the front connection tube member 20 and the breast shield member 10. The connection portions 22, 24, 52, 54 may be as an example and not a limitation, male and female threaded connections or plugs.

The breast shield member 10 is used to connect or attach the concealable and reduced noise breast pump to the breast of the user. In an embodiment, the breast shield member 10 has a lip portion 12 which assists in forming a seal or tight connection with the breast which aids in the pumping or extraction of the user's breast milk. In an embodiment, the breast shield member 10 also has a cup portion 14 which assists in the positioning of the breast shield member 10 over the user's areola portion of the breast with the nipple centered in the cup portion 14.

The fluid capture member 40 is used to capture, hold, and store the breast milk pumped from the user's breast. In an embodiment, the fluid capture member 40 is a bottle or bag. In an embodiment, the fluid capture member 40 can be connected and disconnected from the diaphragm member 30. This may be for example and not a limitation by screwing the fluid capture member 40 on and off with the fluid capture member 40 having a male threaded portion and the diaphragm member 30 having a female threaded portion. This allows the fluid capture member 40 once full to be easily detached from the diaphragm member 30 and stored and another fluid capture member 40 to be attached to the diaphragm member 30 for continuous pumping.

The pump member 60 is used to extract the breast milk from the user and in an embodiment is also comprised of controls to set the pumping strength and frequency for extracting the breast milk. In another embodiment, the pump member 60 is a manual pump, which allows the system to function without the power supply member 70 and the motor member 80.

The power supply member 70 provides the energy or power to operate the motor member 80 which powers the pump member 60. In an embodiment, the motor member 80 and the pump member 60 are one member. In an embodiment, the power supply member 70 are batteries 72.

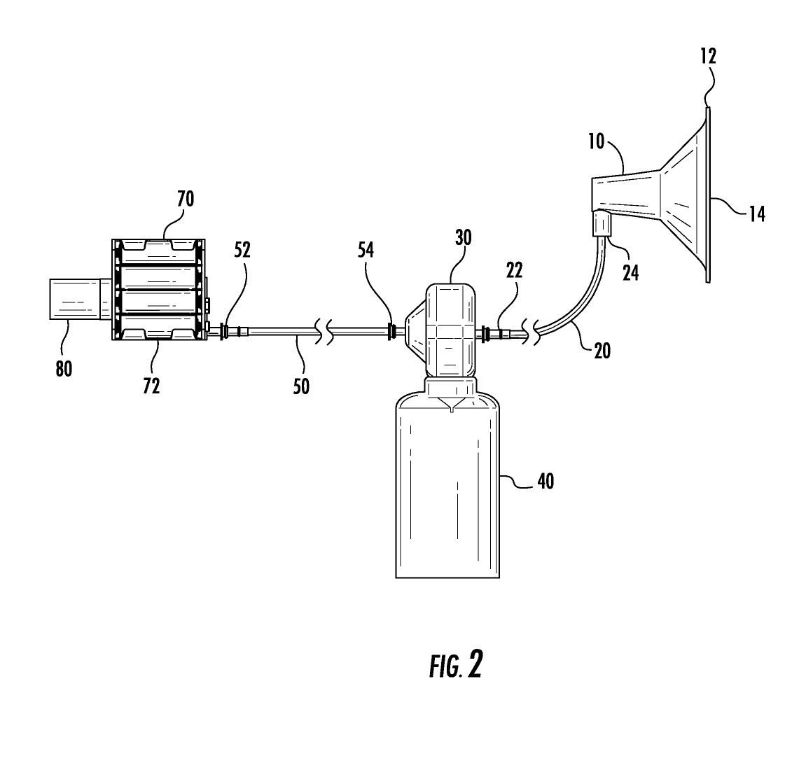

Reference is next made to FIG. 2, a left side view of the concealable and reduced noise breast pump in accordance with the teachings of the present disclosure. In this illustration the concealable and reduced noise breast pump is shown from the left side.

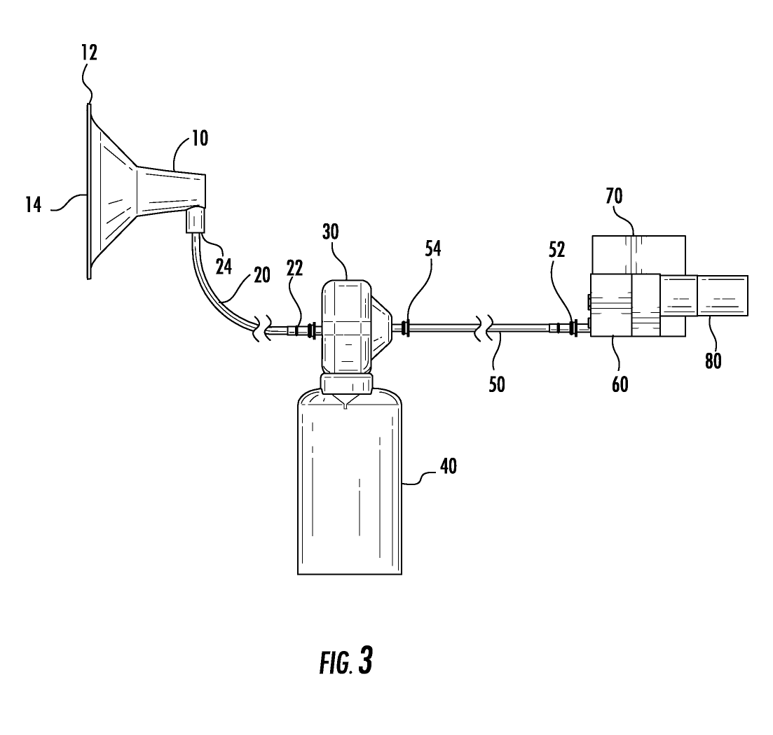

Reference is now made to FIG. 3, a right side view of the concealable and reduced noise breast pump in accordance with the teachings of the present disclosure. In this illustration the concealable and reduced noise breast pump is shown from the right side.

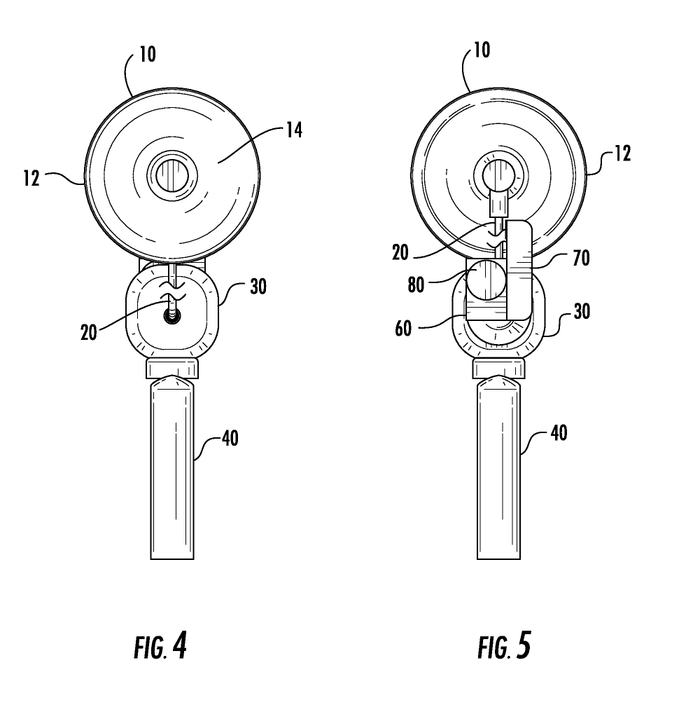

Reference is next made to FIG. 4, a front view of the concealable and reduced noise breast pump in accordance with the teachings of the present disclosure. In this illustration the concealable and reduced noise breast pump is shown from the front.

Reference is now made to FIG. 5, a back view of the concealable and reduced noise breast pump in accordance with the teachings of the present disclosure. In this illustration the concealable and reduced noise breast pump is shown from the back.

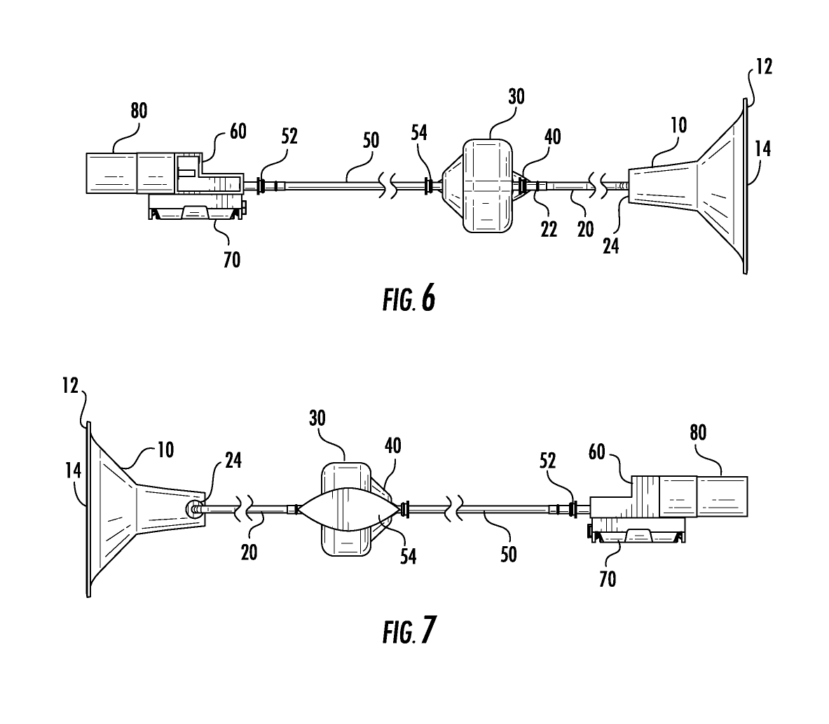

Reference is next made to FIG. 6, a top view of the concealable and reduced noise breast pump in accordance with the teachings of the present disclosure. In this illustration the concealable and reduced noise breast pump is shown from the top.

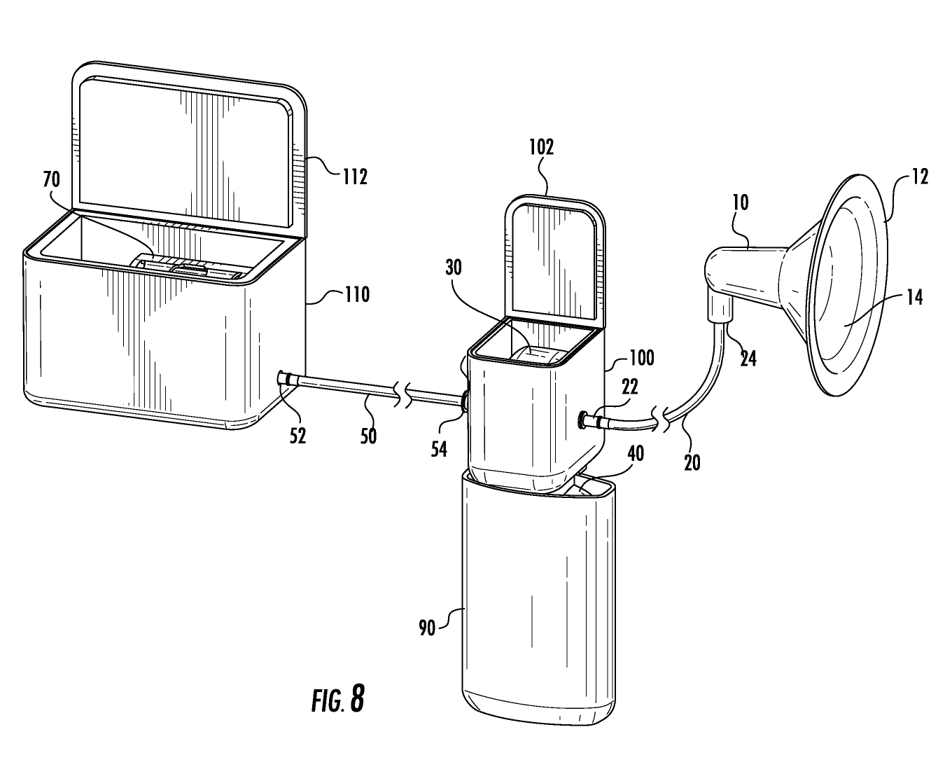

Reference is now made to FIG. 7, a bottom view of the concealable and reduced noise breast pump in accordance with the teachings of the present disclosure. In this illustration the concealable and reduced noise breast pump is shown from the bottom

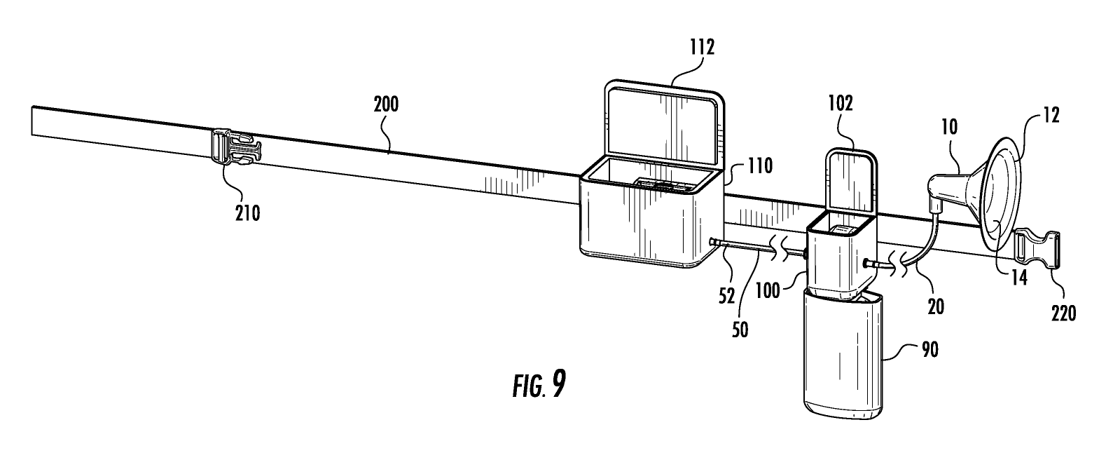

Reference is next made to FIG. 8, a left side perspective view of the concealable and reduced noise breast pump with noise dampening insulation in accordance with the teachings of the present disclosure. In this illustration is an embodiment where the system is further comprised of noise dampening members 90, 100, 110 which cover the diaphragm member 30, the fluid capture member 40, and the pump member 60, the power supply member 70, and the motor member 80 combined to reduce the noise level heard from the concealable and reduced noise breast pump. The noise dampening members 90, 100, 110 are made of noise dampening material such as but not limited to deaden foam. The pump member 60, the power supply member 70, and the motor member 80 noise dampening member 110 has a lid portion 112 in an embodiment. The diaphragm member 30 noise dampening member 100 has a lid portion 102 in an embodiment.

Reference is now made to FIG. 9, a left side perspective view of the concealable and reduced noise breast pump with noise dampening insulation and attachment belt in accordance with the teachings of the present disclosure. In this illustration is an embodiment where the concealable and reduced noise breast pump is further comprised of belt member 200 to be worn around the waist of a user of the system which allows certain members such as the diaphragm member 30, the fluid capture member 40, the power supply member 70, the motor member 80, and/or the pump member 60 to be releasably connected to the belt member 200. In an embodiment the belt member 200 is a neoprene belt having clip portions 210, 200 for attaching and detaching to a user of the concealable and noise reduction breast pump. The means to be releasably connected may be for example but not limited to Velcro or an adhesive. In another embodiment the components are permanently attached to the belt member 200.

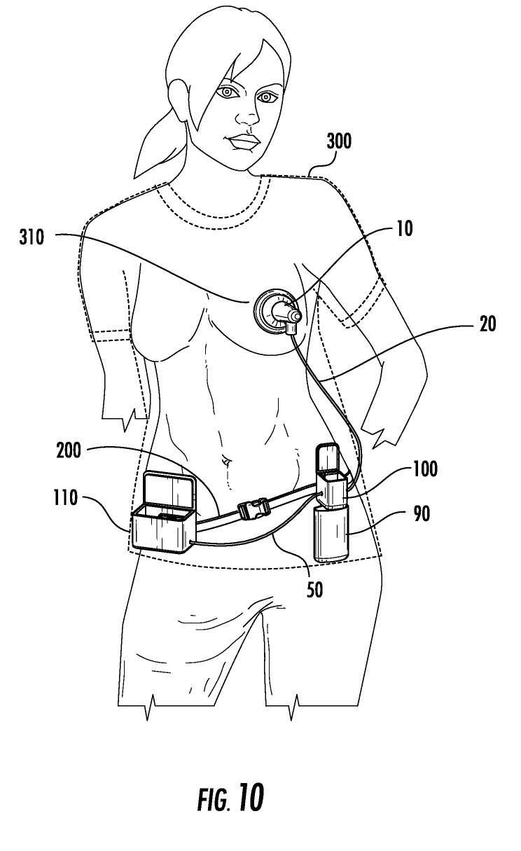

Reference is lastly made to FIG. 10, an environmental view of the concealable and reduced noise breast pump with noise dampening insulation and attachment belt being utilized by a user in accordance with the teachings of the present disclosure. In this illustration the concealable and reduced noise breast pump is shown on a user 300 of the system. The breast shield member 10 is shown on the user's 300 left breast 310. The connection tube members 20, 50 may be sized to fit the user 300 properly. That is a proper length to allow the concealable and reduced noise breast pump to be hidden out of sight and allow the user 300 to move freely without obstruction. Because of the sizing of the components, the concealable and reduced noise breast pump is worn under the user's 300 clothing. In this illustration, it is shown under the user's 300 shirt or blouse. This allows the concealable and reduced noise breast pump to be worn and used in public without detection. In an embodiment, the breast shield member 10 is worn underneath or inside the user's 300 bra. This setup or position holds the breast shield member 10 in position. In addition, the concealable and reduced noise breast pump can be detached at the diaphragm connection portion 22. This allows the breast shield member 10 and the connection tube member 20 to remain hidden and on the user 300. The belt member 200 along with the other components can be removed from the user 300 until breast pumping is desired. Once breast pumping is desired, the belt member 200 along with the other components can be reattached by attaching the belt member 200 to the user 300 and reconnecting the diaphragm member 30 to the connection tube member 20 via the diaphragm connection portion 22.

In brief, the present invention relates to a system and method of use for a concealable and reduced noise breast pump.

The disclosed system and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method, except as may be outlined in the claims.

Consequently, any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The systems and/or methods disclosed and claimed herein can be made and executed without undue experimentation in light of the present disclosure. While the systems and methods of this invention have been described in terms of preferred embodiments, it will be apparent to those skilled in the art that variations may be applied to the systems and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A concealable breast pump comprising:
a breast shield member;
a diaphragm member;
a fluid capture member;
a pump member; and
at least one connection tube member wherein said connection tube members connects
said breast shield member with at least one other said member.

2. The concealable breast pump of claim 1, wherein there are two said connection tube members having one connection tube member joining said breast shield member with said diaphragm member and said second connection tube member connecting said diaphragm member with said pump member.

3. The concealable breast pump of claim 1, further comprising a motor member and a power supply member.

4. The concealable breast pump of claim 3, wherein there are two said connection tube members having one connection tube member joining said breast shield member with said diaphragm member and said second connection tube member connecting said diaphragm member with said pump member.

5. The concealable breast pump of claim 1, wherein said connection tube members are of various lengths to allow proper fitting and placement of the members when in use by an individual.

6. The concealable breast pump of claim 3, wherein said connection tube members are of various lengths to allow proper fitting and placement of the members when in use by an individual.

7. The concealable breast pump of claim 1, wherein said connection tube members are configured to allow elongation and retraction of each said connection tube member.

8. The concealable breast pump of claim 3, wherein said connection tube members are configured to allow elongation and retraction of each said connection tube member.

9. The concealable breast pump of claim 3, wherein said connection tube members are further comprised of connecting portions on at least one end of each said connection tube member.

10. The concealable breast pump of claim 3, wherein said connection tube members are further comprised of connecting portions on each end of each said connection tube member to allow the connection tube members to be releasably attached at their ends.

11. The concealable breast pump of claim 3, wherein said fluid capture member is a bottle.

12. The concealable breast pump of claim 3, wherein said fluid capture member is a bag.

13. The concealable breast pump of claim 3, wherein said fluid capture member is releasably attached to said diaphragm member.

14. The concealable breast pump of claim 3, wherein said pump member is configured with controls allowing a user to set the pumping strength and pumping frequency.

15. The concealable breast pump of claim 3, further comprising noise dampening members.

16. The concealable breast pump of claim 15, wherein said noise dampening members cover said pump member, said power supply member, and said motor member.

17. The concealable breast pump of claim 15, wherein said noise dampening members cover said diaphragm member, said fluid capture member, said pump member, said power supply member, and said motor member.

18. The concealable breast pump of claim 16, wherein said noise dampening members are further comprised of a lid portion.

19. The concealable breast pump of claim 3, further comprising a belt member allowing at least one of the other members to be releasably attached to said belt member.

20. A concealable breast pump comprising:
a breast shield member;
a diaphragm member;
a fluid capture member releasably attached to said diaphragm member;
a motor member;
a power supply member;
a pump member;
two connection tube member having one connection tube member joining said breast shield member with said diaphragm member and said second connection tube member connecting said diaphragm member with said pump member; wherein said connection tube members are configured to allow elongation and retraction of each said connection tube member and wherein each said connection tube member may be of various lengths to allow proper fitting and placement of the members when in use by an individual; wherein said connection tube members are further comprised of connecting portions on at least one end of each said connection tube member;
noise dampening members covering said pump member, said power supply member, and said motor member and wherein each said noise dampening members are comprised of a lid portion; and
a belt member allowing at least one of the other members to be releasably attached to said belt member
